(19) United States
(12) Patent Application Publication
(10) Pub. No.: US 2001/0048936 A1
(43) Pub. Date: Dec. 6, 2001

(54) Gloves and booties for dispensing skin treating agents

(76) Inventor: Melvin B. Prenovitz, Brookline, MA (US)

Correspondence Address: PERKINS, SMITH & COHEN LLP, ONE BEACON STREET, 30TH FLOOR, BOSTON, MA, 02108, US

(21) Appl. No.: 09/748,449

(22) Filed: Dec. 22, 2000

Related U.S. Application Data
(60) Provisional application No. 60/171,836, filed on Dec. 22, 1999.

(51) Int. Cl. A61K 9/70; A61F 5/00

(52) U.S. Cl. 424/443; 602/21

(57) ABSTRACT

A device for dispensing a skin treating agent on an appendage and its method of manufacture. The device having a first side with an inner surface and an outer surface, a second side with an inner surface and an outer surface, the first side and second side connected at an outer seal with the inner surfaces facing one another and forming a chamber for receiving and enclosing the appendage. The first and second sides having a perforated area which when torn apart creates an opening where the appendage is inserted into the chamber and a quantity of skin treating agent distributed in discrete areas on at least one of the inner surfaces of the chamber. A securing member enables the device to be secured to the appendage.

### CROSS REFERENCE TO RELATED APPLICATIONS

This application claims priority of U.S. Provisional Application Ser. No. 60/171,836 filed Dec. 22, 1999 entitled GLOVES AND BOOTIES FOR DISPENSING SKIN TREATING AGENTS.

### BACKGROUND OF THE INVENTION

This invention relates generally to an improved device or article for dispensing lubricants, moisturizers and other medicinal agents to various parts of the body, and, more particularly this invention relates to a glove or bootie, which contains such lubricants, moisturizers and other medicinal agents and its method of manufacture.

Hands and other body appendages of persons readily become chapped and/or react to exposure in adverse environments and therefore require the application of lubricating, moisturizing or medicinal type agents from time to time. Usually, these types of agents are applied to the hands or body appendages when needed, incurring a great inconvenience to the person to whom these agents have been applied. Firstly, these agents must be applied and rubbed into the affected area for a period of time in order to result in the desired absorption necessary to treat the affected areas. Secondly, the application of such agents leaves the appendages with a thin coating of the agent which readily contaminates many materials contacted by the appendage in daily living.

Many different approaches have been used in the past to provide the user with a device for treating appendages of the body. In U.S. Pat. No. 2,601,851, to Jones, an applicator for treating skin ailments is disclosed which utilizes a bag-like envelope for the feet used specifically for the application of medication to treat ringworm or athlete's foot. The device requires breaking of an inner envelope containing the medication by exerting pressure on the foot bag envelope.

Another approach is shown in U.S. Pat. No. 2,916,036 to Sutton, which discloses rubber gloves having an elaborate composition of layers or holding areas containing globules therein such that the warmth of the hand to which the gloves are applied may allow some lanolin to escape down the fibers of the glove to the skin of the user.

Cahill, U.S. Pat. No. 3,116,732, discloses a disposable hand care glove comprising an outer layer of leak-proof material, an inner layer of porous material and a plurality of reservoirs for containing a hand-treatment medication. In order to use this device, the reservoirs must be ruptured.

Another approach is shown by Charos, U.S. Pat. No. 3,342,182. This patent discloses a packaged cream applicator glove which fits tightly around a person's wrist, has inner and outer portions, and a plurality of cartridges for holding the cream which extend through slots in one of the outer panel portions. Again, pressure must be placed over the cartridges in order to massage the cosmetic preparation into the skin of the hand.

Various methods are also known by which these prior art gloves are provided with various treatment medications. For example, U.S. Pat. No. 3,384,083 to Cozza et al, discloses a plastic glove manufactured from a continuous length of heat sealable fiber upon which a medicant is sprayed by a printing device. A second layer of heat sealable film is superimposed over the first layer, in order to form an article of apparel.

Berry patent No. 5,869,072 is a manufacturing method incorporating a porous lining to dispense lotion over time. DeFina patent No. 5,614,202 uses a porous inner material lining that dispenses lotion through activation of heat from the hand, or appendage. Mast patent No. 5,891,116 is a method of treating the hand with a wax composition in an elastic glove.

It is readily apparent that such past methods of dispensing skin treating agents (1) lack the capability to prevent water moisture evaporation during non usage over long periods of time, (2) lack the capability of maintaining a natural herbal treating agent for long period of time during non usage without the use of non-natural preservatives, (3) lack the ease of adhering the dispenser to the part of the body being treated, and (4) lack the ability to create predetermined patterns for the dispensed lotions.

In view of the foregoing, it should be apparent that there still exists a need in the art for an article of apparel which can be readily used to apply a lubricating, moisturizing or medicinal type agent to the hand or other part of the body in a quick, clean and efficient manner and a method to preserve the agent without using chemical preservants.

It is, therefore, a primary object of the present invention to provide an article of apparel for delivering a lubricating, moisturizing or medicinal type agent to the hand or other part of the body.

It is a further object of this invention to provide an article of apparel that will deliver a lubricating, moisturizing or medicinal type agent to the hand or other part of the body for an extended period of time.

It is another object of this invention to provide a simple method for applying lubricating, moisturizing or medicinal agents to parts of the human body such that the user may go about his/her daily routine without being concerned about contaminating materials touched by the portion of the body being treated.

It is still another object of the present invention to provide an efficient and inexpensive means for applying lubricating, moisturizing or medicinal type agent to a desired part of the body.

It is an even further object of the present invention to provide an article of apparel which utilizes the heat of the area of the body being treated to enhance the absorption of the lubricant, moisturizer or medicinal agent being applied to the affected area.

It is still another object of the invention to provide a method of packaging medication-filled dispensers to extend their shelf-life.

### SUMMARY OF THE INVENTION

The objects set forth above as well as further and other objects and advantages of the present invention are achieved by the embodiments of the invention described hereinbelow.

The articles of apparel making up the present invention are generally in the form of a glove and/or bootie for applying lubricating, moisturizing or medicinal type agent to a desired part of the body which is sealed on all sides to:

1) prevent fluids from escaping,

2) prolong the shelf life of its contents, and

3) allow preservative free ingredients to be used,or to reduce the reliance on chemical based preservatives.

The articles of apparel are perforated, scored, or use other means to allow the material such as plastic to tear along a preformed line at the wrist, ankle, or elsewhere, to peel open, or tear open in order to insert appendage.

The articles of apparel, usually in the form of a glove or bootie, are secured to the body by means of a tab having an adhesive backing, or the part that tears free. In one scenario the tab is placed at the outer edge of the article in the vicinity of the tear line creating a square or rectangular appendage to the glove. A two-sided adhesive tape is applied to the tab with one side adhering to the article. A peel-off covering on the outer layer of the tape exposes a sticky adhesive allowing the tab to be wrapped around the wrist or ankle to secure the article from slippage. In a second arrangement, the tear part can be partially left attached at one side of the wrist or ankle. The far-end seal holding the loose end of the torn section can be split apart at the seam and tied together, or it creates a strap to wrap around the wrist, ankle, etc. preventing the glove or bootie from slipping off. The loose straps can be secured by means of hooks, holes, loops, adhesives, a Velcro® type fabric, etc. that can be designed into the glove to mate with the strap.

In use, the glove or bootie, which already contains the treating agent, is then placed over the affected portion of the body and the treatment process begins. The gloves or bootie may contain a lubricating, moisturizing or medicinal type agent to be applied to the body.

In addition, the article of apparel is manufactured by a process which permits the above-mentioned benefits of the invention to be readily obtained. After manufacture, the glove and bootie may be packaged individually, in a package of multiple gloves, or bulk in a dispenser box or container. They may also be packaged in a sterilized or non-sterilized container.

For a better understanding of the present invention, together with other and further objects thereof, reference is made to the accompanying drawings and detailed description.

### BRIEF DESCRIPTION OF THE DRAWINGS

FIG. 1 is a pictorial top view of the glove of the present invention;

FIG. 2 is a pictorial side view of the bootie of the present invention;

FIG. 3 is a pictorial view of a hand positioned inside the glove of the present invention;

FIG. 4 is a pictorial view of a foot inside the bootie of the present invention;

FIG. 5 is a pictorial view of the lines, or beads, of agent placed within the glove of the present invention; and

FIGS. 6a-6d are pictorial views illustrating the manufacturing method of this invention to apply agents to the film and to retain the lines within the enclosed chamber using special rollers.

### DETAILED DESCRIPTION OF THE PREFERRED EMBODIMENTS

The glove or bootie of the present invention is utilized to cover the affected portion of the body in a safe and simple manner. Neither the dispensing glove nor bootie requires additional structure to affect the containment of the treating agent.

The glove or bootie is sealed with an easy open arrangement that can also serve as a restraining device, and utilized to cover the affected portion of the body in a safe and simple manner for an extended period of time. Neither the dispensing glove nor bootie requires additional structure to affect the containment of the treating agent. The glove or bootie also utilizes the heat of the area of the body being treated to enhance the absorption of the lubricant, moisturizer or medicinal agent being applied to the affected area. This confines the treating agent to the treated part, and, therefore, decreases any losses to evaporation or juxtaposed garments, bed sheets, work apparel, etc., as compared to the mere application of the treating agent to the affected part of the body. The present invention also provides a method of packaging the medication-filled apparel to extend their shelf-life.

The articles of apparel, usually in the form of a glove or bootie, are secured to the body by means of a tab having an adhesive backing, or a part that tears free. In one scenario the tab is placed at the outer edge of the article in the vicinity of the tear line creating a square or rectangular appendage to the glove. A two-sided adhesive tape is applied to the tab with one side adhering to the article. A peel-off covering on the outer layer of the tape exposes a sticky adhesive allowing the tab to be wrapped around the wrist or ankle to secure the article from slippage. In a second embodiment, the tear part can be partially left attached at one side of the wrist or ankle. The loose end of the torn section can be split apart at the seam creating a strap to wrap around the wrist, ankle, etc. to prevent the glove or bootie from slipping off. The loose straps can be secured by tying a knot, or by means of Hooks, holes, loops, adhesives, a Velcro® type fabric, etc that can be designed into the glove to mate with the strap.

The article of this invention, typically a glove or bootie, retains heat and humidity naturally expelled through the skin creating a sauna environment for the encased appendage. The pores of the skin open accelerating deep absorption of the lubricants, moisturizers and other medicinal agents into the skin and cuticles.

As a result of the manufacturing process of this invention, the present invention provides an exact measurement of lubricants, moisturizers and other medicinal agents necessary to moisturize or heal the enclosed appendage. In a professional environment, it avoids waste of applying more material than is necessary to achieve the desired results.

The completely sealed glove, bootie, etc. retards moisture evaporation of lubricants, moisturizers and other medicinal agents inserted inside it. The ingredients remain fresh until each article is individually opened. The completely sealed article also prevents bacteria from entering the article, thus ensuring sanitary storage.

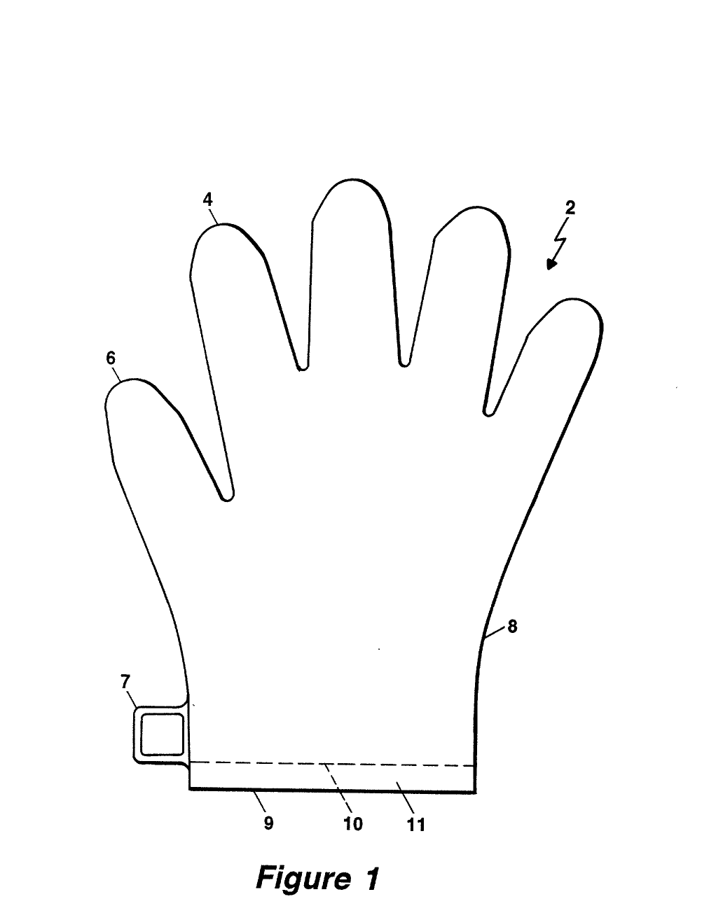

Referring now in detail to the drawings wherein like parts are designated by like reference numerals throughout, there is illustrated in FIG. 1 a dispenser glove 2 of the present invention having four fingers 4 and a thumb 6. This glove 2 has a hollow lumen into which a medicinal type agent, lubricant or moisturizer, may be placed during or prior to packaging as more clearly described below. The glove 2 is preferably of the single layer or thickness polyethylene or polypropylene type, and may be reversible.

The film chosen for use in this invention, after extensive testing, is in the form of commercially available olefin films. The primary attributes required were a film product that was able to deliver a soft hand (low modulus, very tactile), high tear strength in both machine and cross directions, high dart impact strength, good clarity, good MVTR barrier properties, ability to deliver seal integrity through therapeutic coatings within glove profile, chemically inert to most consumer emollients and oils.

The film selected is manufactured from a specially formulated, rapid-cooled blown polypropylene film extrusion process, manufactured by Tufpak, Inc. of Ossipee, NH—registered trademark SUPROP type PP-05 clear. The unique nature of how the film was cooled immediately after extrusion, delivered a product that had a measurably higher amorphous to crystalline ratio than what could be achieved from a typical air-cooled extrusion process. The resultant desired physical properties, therefore, were correspondingly unique and met the inventive requirements for strength, flex-crack resistance, clarity, pliability, sealing through film that is coated with lotion, and superior moisture barrier protection compared to low density polyurethane.

When placed on the hands, this glove 2 will deliver the lubricating/moisturizing agent to the hands by contact either for an overnight period or for a shorter wearing time. This treatment acts to moisturize the skin and cuticles of the user. This glove may also contain any other medicinal type agent, either prescription or non-prescription, especially of the ointment, cream or lotion varieties, for the purpose of delivering these agents in the same manner as that discussed immediately above. Examples of this type of medicinal agent are: arthritis medicine and the accompanying warmth thereof; moisturizers; topical antibiotics; and burn medicine.

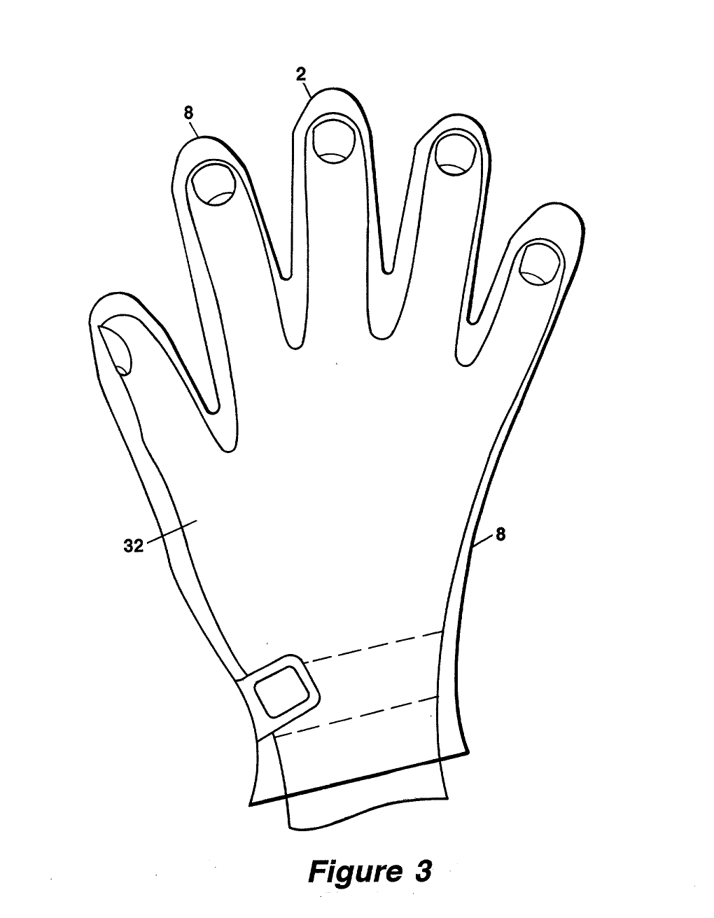

The entire outside edge of the glove 8 provides a heat seal. This sealing process is performed during the manufacturing process when the glove is die cut. The entire outer edge, including the wrist area 9 where a hand will be inserted, is sealed. Approximately one half inch (½″) from the outer edge where a hand will insert, a perforated, scored, or other means to allow plastic to tear along a pre-formed line 10 at the wrist or elsewhere, to peel open, or tear open in order to insert appendage. It might be a straight line across the wrist area, or it may be offset to accommodate a variety of alternate securing methods. The part that tears free 11 can be partially attached at one end creating a strap to wrap around the wrist, ankle, etc. preventing the glove from slipping off. Hooks, holes, loops, etc can be designed into the glove to mate with the strap or as preferred and shown in FIG. 3 a tab 7 is placed at the outer edge of the article in the vicinity of the tear line creating a square or rectangular appendage to the glove. A two-sided adhesive tape is applied to the tab with one side adhering to the article. A peel-off covering on the outer layer of the tape exposes a sticky adhesive allowing the tab to be wrapped around the wrist or ankle to secure the article from slippage. The method of securing the article to the hand or foot can be accomplished with one hand.

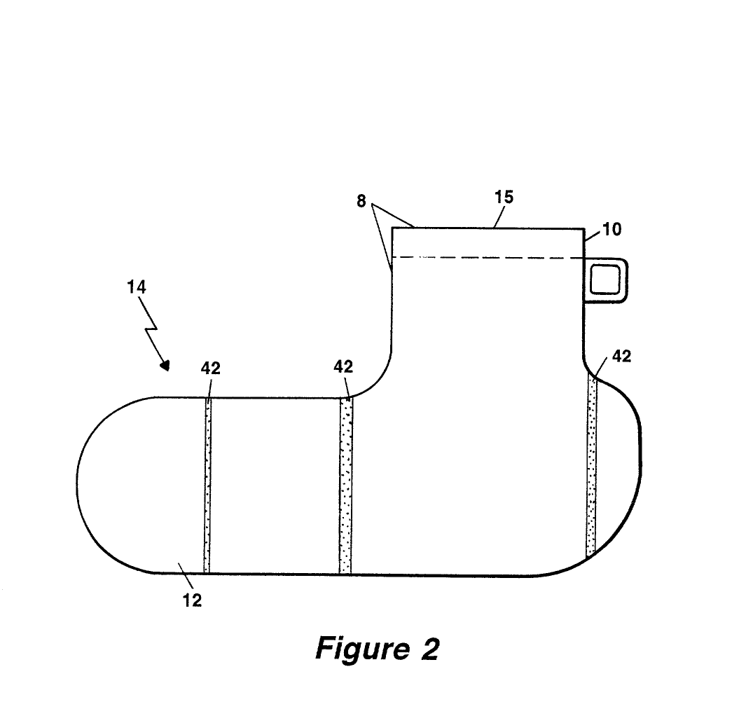

FIG. 2 discloses the same embodiments of the glove in FIG. 1 as it relates to a bootie 14. A chamber 12 is created by the bootie 14 for insertion of a foot. The entire outer seam 8 of the bootie is sealed. Approximately one half inch (½″) from the outer edge 8 where a foot will insert, a perforated, scored, or other means to allow plastic to tear along a pre-formed line 10 at the ankle, or elsewhere, to peel open, or tear open in order to insert appendage. It might be a straight line across the ankle area 15, or it may be offset to accommodate a variety of alternate securing methods.

FIG. 3 demonstrates an adhesive tab wherein the perforated portion 11 of the glove 2 is removed and a tab 7 (see also FIG. 1) adjacent to the opening of the glove or bootie wraps around the wrist or ankle.

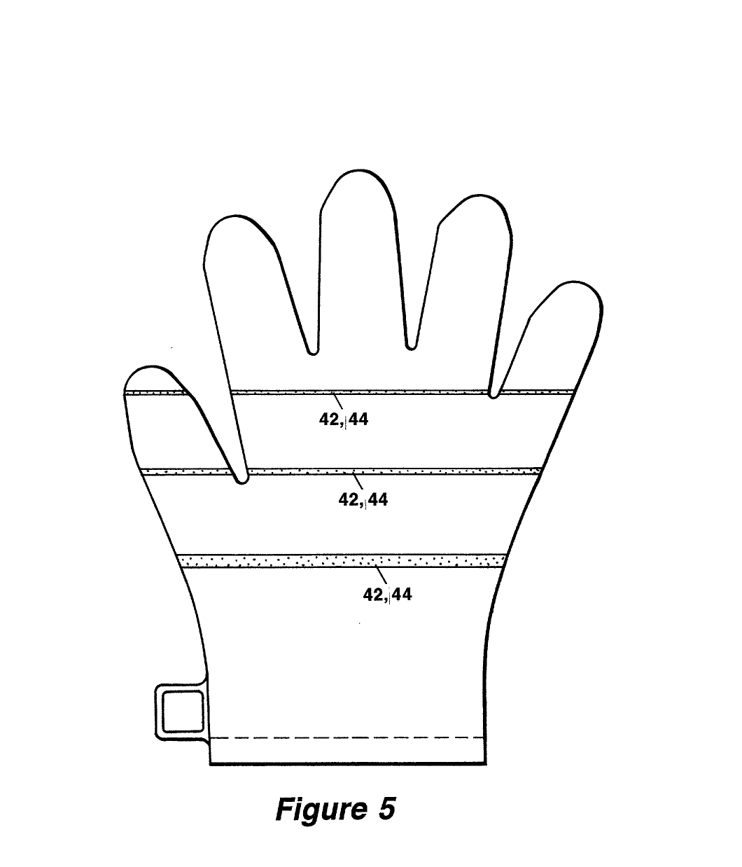

More specifically, FIG. 3 shows the glove 2 with a hand 32 inserted into it. Lotion in the form of beads 45 or lines 44 (see FIG. 5) that was inserted in the glove 2 at manufacture forming a lining 39 inside is dispensed onto the hand 32 as the hand 32 is placed into the glove 2. The outer seal 8 of the glove 2 retains the hand 32 and lotion. The tab 7 is used to secure the glove 2 in place as described above. In the alternative, the perforated portion of the glove 2 can be used to form a strip loop which can be looped around the wrist and secured in place.

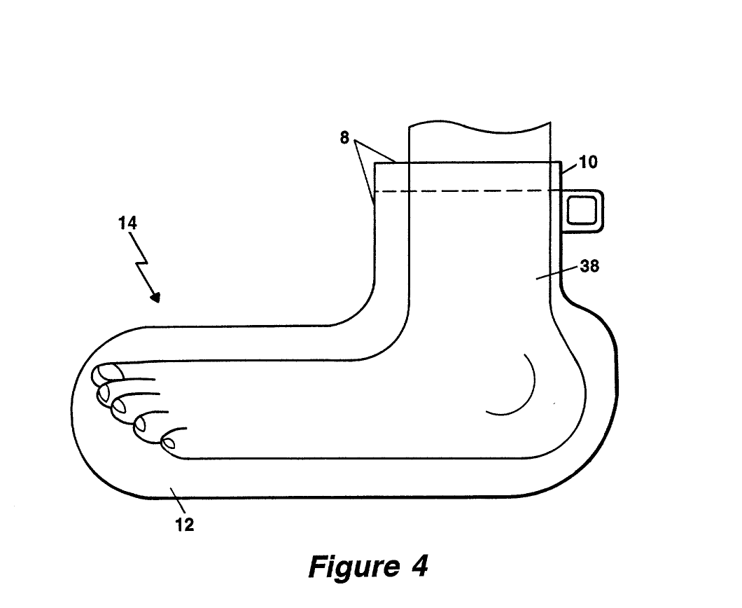

FIG. 4 shows the bootie 14 with a foot 38 inserted into it. Lotion 34 that was inserted in the bootie 14 at manufacture forming a lining 39 inside is dispensed on to the foot 38 as the foot 38 is placed into the bootie 14. The outer seal 8 of the bootie 14 retains the foot 38 and lotion 34. A same manner of attachment as with the glove 2 can be used for the bootie 14 as well.

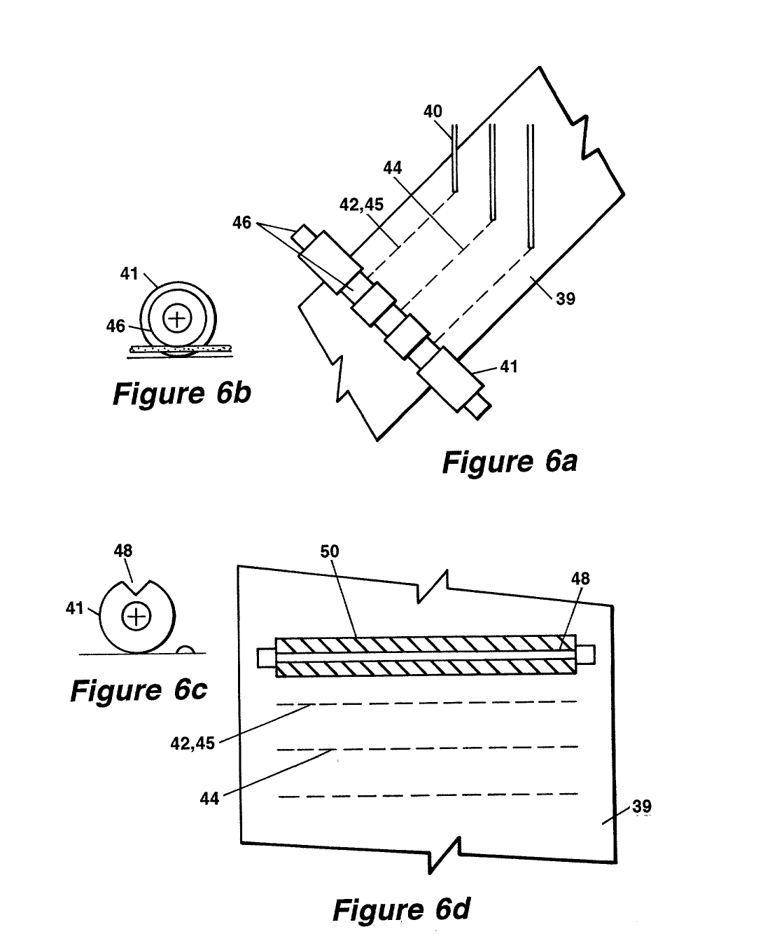

FIG. 5 demonstrates the method in which agents 42 are applied to the film 39 of the article of apparel. A series of pens or valves 40 as shown in FIG. 6a dispense agents 42 onto the film 39 travelling beneath them. The pens 40 may dispense a continuous line 44 of agent 42 or intermittent drops 45 creating beads along the same plane.

Rollers 41, used to flatten, align and transport the film 39 through the manufacturing process are grooved at 46 as shown in FIG. 6a and 6b at the exact location where the line 44, or bead 45, of agent 42 passes by 46 preventing the agents 42 from flattening and spreading over the entire surface of the film 39. A V-shaped groove 48 as shown in FIGS. 6c and 6d is machined or designed into the rollers 41. The grooves may run in a parallel line to the rotation of the rollers 41 (see FIGS. 6a and 6b) in which case the lotion will travel in a continuous path through the grooved portion, or the grooves may run lengthwise 50 (see FIGS. 6c and 6d) perpendicular to the roller's rotating motion skipping over the beads 45, or lines 44, as they pass through.

Stated more succinctly, the present invention as described above utilizes a method of inserting lotions, moisturizers, and other medicinal agents to the inside of the articles. The agents 42 will be laid down in individual beads 45 or continuous lines 44 traversing a straight line that could be horizontal, vertical, or diagonal along the internal surface of the article. This is considerably different than spreading the ingredients out over the entire inner surface. In the case of a completed glove 2, one bead 45, or line 44, of lotion 34, etc. may horizontally traverse the glove 2 just below the opening to the four fingers 4. A second bead, or line, may horizontally traverse the glove 2 from the opening below the thumb to a midway point at the opposite side of the palm. A third bead, or line, may horizontally traverse where the wrist and hand meet. A similar pattern is utilized for booties and other articles of apparel.

As the two sides of the article (e.g.glove) press or form together, the internal lotion, moisturizers, etc. will be spread out to a width of approximately ½″ wide, however, a range of ¼″ to 1″ is also acceptable under certain circumstances. Definitive lines preferably will be created where each line of lotion begins and ends. The volume of ingredients in each line will vary to its respective proximity to the sequence that the appendage comes into contact with it. The outermost bead/line that contacts the appendage first will have the most volume. The second bead/line will have slightly less, and the innermost or third bead/line will have the least volume. The appendage will be picking up and carrying the ingredients along with it as it is inserted into the article.

As clearly described above, the manufacturing process of this invention which achieves the above effect relies upon the lotions, etc. being pumped through multiple dispensing heads strategically positioned as one layer of the film travels by. The rollers built into the manufacturing machine to flatten, align and transport the film through the manufacturing process are grooved at the bead, or line, locations to prevent the lotions from spreading out along the entire inner surface of the article. The grooved rollers ensure the beads, or lines, remain intact until they are spread by the pressure of the two sides of the article coming into contact with each other. Different rollers are required for gloves, booties, and other articles. The grooves may run in a parallel line to the rotation of the rollers, in which case the lotion will travel in a continuous path through the grooved portion, or the grooves may run lengthwise perpendicular to the roller's rotating motion skipping over the beads, or lines, as they pass through.

Any of the dispensers or articles of apparel of the present invention may be either individually packaged and wrapped or may be packaged collectively in a single container such as a multi-pair pouch or as pop-up pre-medicated gloves in a box. The dispensers may be of one or varied sizes.

There has thus been disclosed a simple, yet effective, dispenser for containing lubricating, moisturizing or other medicinal agents for application to parts of the body. No additional structures need be contained in, on or accompany the dispenser gloves or booties of the present invention. In addition, these dispensers or articles of apparel may be utilized on non-human mammals and other animals.

Although only a preferred embodiment is specifically illustrated and described herein, it will be appreciated that other modifications and variations of the present invention are possible in light of the above teachings and within the purview and spirit and intended scope of the claimed invention.

What is claimed is:

1. A device for dispensing a skin treating agent on an appendage comprising:
a first side with an inner surface and an outer surface;
a second side with an inner surface and an outer surface, said first side and second side connected at an outer seal with the inner surfaces facing one another and forming a chamber for receiving and enclosing the appendage, the first and second sides having a perforated area which when torn apart creates an opening where the appendage is inserted into the chamber;
a quantity of skin treating agent distributed in discrete areas on at least one of the inner surfaces of the chamber; and
a securing means which enables the device to be secured to the appendage.

2. The device of claim 1 wherein said device is a glove.

3. The device of claim 2 wherein said discrete areas comprise a plurality of strips of said skin treating agent, each of said strips being of a different volume, the volume of each of said strips decreasing in size in direct proportion to its distance from said opening.

4. The device of claim 3 having at least three strips of said skin treating agent.

5. The device of claim 1 wherein said device is a bootie.

6. The device of claim 5 wherein said discrete areas comprise a plurality of strips of said skin treating agent and wherein said strip located at the greatest distance from said opening being the least volume.

7. The device of claim 6 having at least three strips of said skin treating agent.

8. The device of claim 1 wherein at least a portion of said securing means is affixed adjacent to said opening.

9. The device of claim 1 wherein the skin treating agent contains substantially no preservative.

10. The device of claim 1 wherein said first side and second side are comprised of a material selected from the group consisting of polyethylene and polypropylene.

11. A method of manufacturing a device for dispensing a skin treating agent on an appendage comprising:
providing a first film of material with an inner surface and an outer surface;
providing a second film of material with an inner surface and an outer surface;
dispensing the skin treating agent on the inner surface of at least one of the films of material in discrete areas;
placing the other film of material on top of the one film with the inner surfaces facing one another, forming a two film assembly;
providing a mechanism for transporting the assembly and maintaining the skin treating agent in discrete areas; and
joining the first film to the second film wherein the skin treating agent is maintained within a chamber between the films.

12. The method of claim 11 wherein the skin treating agent is dispensed by a plurality of dispensers.

13. The method of claim 11 wherein the mechanism for transporting the assembly comprises a roller having grooves therein.

14. The method of claim 13 wherein the grooves are positioned along a longitudinal axis of the roller.

15. The method of claim 13 wherein the grooves are positioned transverse to a longitudinal axis of the roller.
